(19) United States
(12) Patent Application Publication
(10) Pub. No.: US 2023/0290451 A1
(43) Pub. Date: Sep. 14, 2023

(54) MEDICAL DATA STORAGE AND RETRIEVAL SYSTEM AND METHOD THEREOF

(71) Applicant: DATABILITY LTD., Rosh Ha'Ayin (IL)

(72) Inventors: Maor FARID, Rosh HaAyin (IL); Mordechai MORAVIA, Rosh HaAyin (IL)

(21) Appl. No.: 17/974,856

(22) Filed: Oct. 27, 2022

(30) Foreign Application Priority Data
Mar. 14, 2022 (IL) 291370

(51) Int. Cl. G16H 10/60

(52) U.S. Cl. CPC G16H 10/60

(57) ABSTRACT

A computerized method for generating a data storage including medical data is provided. The method includes, by a processor and a memory circuitry, obtaining a plurality of data items, wherein the data items comprise medical data pertaining to a patient, constituting patient medical data, wherein the patient medical data includes at least two different medical data types; processing the plurality of obtained medical data to generate a unified representation of the patient medical data; and storing data indicative of the generated unified representation in the database. There is also provided a computerized method for providing medical data by receiving medical data pertaining to a patient and conducting a search in a database for identifying stored similar data.

### CROSS REFERENCE TO RELATED APPLICATIONS

This application claims the benefit of priority of Israeli Patent Application No. 291370, filed Mar. 14, 2022, the contents of which are all incorporated herein by reference in their entirety.

### TECHNICAL FIELD

The presently disclosed subject matter relates to storage and retrieval of medical data and, more particularly, to storage and retrieval of medical data in a manner that enables to search and retrieve medical data in an efficient and more precise manner.

### BACKGROUND

While the average life duration is increasing annually, the amount of medical data gathered for patients is also rapidly increasing. Medical data sources are varied, and include various type of data pertaining to patients, such as records of patients with details describing patient parameters, diseases, their stages, treatments, lab tests, and more. Also, technological development in the imaging devices industry now enables to capture imaging data while constantly improving resolution, speed, and efficiency, which contribute to the amounts of visual clinical data that is available. As a result, more and more clinical data is generated in hospitals, medical centers, and wearable medical devices.

When professionals such as clinicians/radiologists face a clinical case of a new patient, they tempt to apply their prior experience and knowledge to diagnose the pathology associated with the patient's symptoms, as presented to them. They use both the patient scan or series of scans and his\her medical background, clinical history, and lab results. These create a full clinical picture that is used for forming a proper diagnostic, assessing the possible consequences, and establishing a personalized treatment plan.

However, the aforementioned process is becoming more complex due to the ever-growing amount of medical data that exists for each patient, when trying to search medical data, causing the diagnosing doctor to overlook important information due to strict time limitations that exist in medical systems around the world.

Also, the tremendous amount of medical data could be used by professionals to treat others. However, searching the medical data sources is not always feasible, as the medical data is not easily accessible, and, even if data repositories exist, they pertain to specific types of medical data, such as only imaging data, without any relation to other medical data of the patient.

Hence, it is required to enable the accessibility of the various medical sources to professionals in a more efficient manner.

Some current diagnostic systems make use of artificial intelligence (AO-based algorithms. However, AI algorithms tend to be of black-box nature, and are thus unexplainable. The latter is extremely problematic for clinical systems and real-time decision-making, in which mere diagnosis is provided, without explicit explanation of the reasons for the diagnosis. In such cases, professionals, seeking to retrieve further information pertaining to the diagnosis, remain unable to retrieve such. Also, medicolegal wise, black-box nature solutions tend to be more problematic.

Moreover, current development of AI diagnostic systems is aimed at tailor-made solutions for each pathology, resulting in very costly and time-consuming solutions, while not allowing full scalability for a broader scope of possible pathologies.

### GENERAL DESCRIPTION

According to one aspect of the presently disclosed subject matter there is provided a computerized method for generating a data storage including medical data, the method comprising by a processor and a memory circuitry:
obtaining a plurality of data items, wherein the data items comprise medical data pertaining to a patient, constituting patient medical data, wherein the patient medical data includes at least two different medical data types;
processing the plurality of obtained medical data to generate a unified representation of the patient medical data; and
storing data indicative of the generated unified representation in the database.

In addition to the above features, the system according to this aspect of the presently disclosed subject matter can comprise one or more of features (i) to (xi) listed below, in any desired combination or permutation which is technically possible:
(i). wherein the processing is done using one or more AI models.
(ii). wherein the patient medical data comprises at least two of: medical records including unstructured or structured data, 2D or 3D medical imaging data, medical tests, patient history, a doctor's patient summary, patient's clinics summary, or a combination thereof.
(iii). wherein generating the unified representation further comprises: determining the medical data types of the patient medical data; for each determined data type, selecting a respective AI model to execute on the patient medical data; for each determined data type, executing the selected respective AI model to generate a feature vector, resulting in a plurality of generated feature vectors; fusing the generated feature vectors to generate a unified representation of the patient medical data.
(iv). wherein the AI models are selected from a group comprising at least: Convolutional Neural Network (CNN) backbone, Fully Connected Network (FCN), and NLP (Natural Language Processing) backbone.
(v). wherein fusing the generated feature vectors is performed by an AI fusion model.
(vi). wherein the method further comprises: processing the generated unified representation, using an AI model, to generate a similarity vector, wherein the similarity vector is indicative of key features of the patient medical data; associating generated similarity vector with the unified representation; and storing the generated similarity vector.
(vii). wherein the method further comprises: indexing the similarity vector to facilitate retrieval of the medical data from the memory.
(viii). wherein indexing the similarity vector further comprises: associating the similarity vector with one or more predefined searchable data fields from the patient medical data.
(ix). wherein indexing the similarity vector further comprises: based on the similarity vector, generating a lower-dimension searchable vector; and associating the generated lower-dimension searchable vector with the similarity vector.
(x). wherein the method further comprises: processing the generated unified representation, using an AI model, to generate a similarity vector, wherein the similarity vector is indicative of key features of the patient medical data; associating generated similarity vector with the unified representation; and storing the generated similarity vector.
(xi). wherein the method further comprises: obtaining additional medical information not pertaining to a specified patient; generating a unified representation of the additional medical information; and storing the generated unified representation in the memory.

According to another aspect of the presently disclosed subject matter there is provided a computerized system for generating a data storage including medical data, the system comprising a processing and memory circuitry (PMC) configured to:
obtain a plurality of data items, wherein the data items comprise medical data pertaining to a patient, constituting patient medical data, wherein the patient medical data includes at least two different medical data types;
process the plurality of obtained medical data to generate a unified representation of the patient medical data; and
store data indicative of the generated unified representation in the database.

According to another aspect of the presently disclosed subject matter there is provided a non-transitory computer readable storage medium tangibly embodying a program of instructions that, when executed by a computer, cause the computer to perform a method for generating a data storage including medical data, the method comprising, by a processor and a memory circuitry:
obtaining a plurality of data items, wherein the data items comprise medical data pertaining to a patient, constituting patient medical data, wherein the patient medical data includes at least two different medical data types;
processing the plurality of obtained medical data to generate a unified representation of the patient medical data; and
storing data indicative of the generated unified representation in the database.

The system and the non-transitory computer readable storage medium disclosed in accordance with the aspects of the presently disclosed subject matter detailed above can optionally comprise one or more of features (i) to (xi) listed above with respect to the method, mutatis mutandis, in any technically possible combination or permutation.

According to another aspect of the presently disclosed subject matter there is provided a medical data storage and retrieval system for a computer having a processing and memory circuit (PMC), comprising:
a processor of the PMC for configuring the memory of the PMC to store medical data, wherein the medical data comprises:
a plurality of unified representations,
wherein each unified representation is associated with medical data pertaining to a patient, constituting patient medical data, and was generated based on a plurality of data items, wherein the data items comprise the medical data, wherein the medical data includes at least two different medical data types.

In addition to the above features, and to features (i) to (xi), the medical data storage and retrieval system according to this aspect of the presently disclosed subject matter can comprise one or more of features (a) to (j) listed below, in any desired combination or permutation which is technically possible:
(a) wherein each unified representation is generated using one or more AI models.
(b) wherein the patient medical data comprises at least two of: medical records including unstructured or structured data, 2D or 3D medical imaging data, medical tests, patient history, a doctor's patient summary, patient's clinics summary, or a combination thereof.
(c) wherein each of the unified representations is generated by: determining the medical data types of the patient medical data; for each determined data type, selecting a respective AI model to apply on the patient medical data; for each determined data type, applying the selected respective AI model to generate a feature vector, resulting in a plurality of generated feature vectors; fusing the generated feature vectors to generate the unified representation of the patient medical data.
(d) wherein the AI models are selected from a group comprising at least: Convolutional Neural Network (CNN) backbone, Fully Connected Network (FCN), and NLP (Natural Language Processing) backbone.
(e) wherein fusing the generated feature vectors is performed by an AI fusion model.
(f) wherein each of the unified representations is associated with a respective similarity vector, wherein each similarity vector is generated from the unified representation, using an AI model, and is indicative of key features of the patient medical data.
(g) wherein the similarity vectors are indexed to facilitate retrieval of the medical data from the memory.
(h) wherein each similarity vector is associated with one or more predefined searchable data fields from the patient medical data.
(i) wherein each similarity vector is associated with a generated lower-dimension searchable vector.
(j) wherein the medical data further comprises: a plurality of unspecified patient unified representations; wherein each unspecified patient unified representation is associated with additional medical information not pertaining to a specified patient, and is generated based on the medical information not pertaining to a specified patient, wherein the additional medical information includes at least two different medical data types.

According to another aspect of the presently disclosed subject matter there is provided a computerized method for providing medical data, the method comprising:
receiving data indicative of a first medical data pertaining to a first patient, wherein the first medical data includes at least two different medical data types;
generating a unified representation of the received first medical data;
based on the generated unified representation, conducting a search in a database for identifying stored unified representations that are similar to the generated unified representation, according to a similarity criterion, wherein at least one unified representation of the stored unified representations is associated with a second patient, and is generated based on second medical data of the second patient, wherein the second medical data include at least two different medical data types;
identifying at least one similar unified representation;
obtaining the medical data associated with the least one similar unified representation; and
providing the obtained medical data.

In addition to the above features, to features (i) to (xxiii), and to features (a) to (j), the method according to this aspect of the presently disclosed subject matter can comprise one or more of features (1) to (10) listed below, in any desired combination or permutation which is technically possible:
(1) wherein identifying the similar unified representations further comprises: for each stored unified representation of the plurality of stored unified representations: calculating a distance between the generated unified representation and the stored unified representation; and determining that the stored unified representation meets the similarity criterion in response to the calculated distance not exceeding a pre-configured threshold.
(2) wherein the identified stored unified representations that are similar to the generated unified representation are indicative that at least one parameter associated with the stored medical data is pathology similar to at least one parameter associated with the first medical data.
(3) wherein providing the associated medical data further comprises providing a respective similarity degree, calculated based on the distance, for each of the at least one identified similar unified representation.
(4) wherein prior to obtaining the stored medical data, the method further comprises: filtering out at least one identified similar unified representation based on medical heuristics; obtaining the stored medical data of similar unified representation which were not filtered out; and providing the obtained non-filtered out stored medical data.
(5) wherein prior to obtaining the stored medical data, the method further comprises: determining a priority for the at least one identified similar unified representation, based on medical heuristics; and providing the obtained medical data according to the determined priority.
(6) wherein receiving the data indicative of a first medical data further comprises: receiving a region of interest (ROI) input; and generating the unified representation based on the received ROI.
(7) wherein at least two similar unified representations are identified, wherein the identified similar unified representations are respectively associated with stored first and second medical data of first and second patients, and wherein the method further comprises: applying statistical methods to identify at least one pattern among the first and second medical data;
calculating a respective probability for each identified at least one pattern;
and provide at least the pattern having a highest probability.
(8) the method further comprising, for each of the at least one calculated probability: providing the identified pattern, in response to the probability meeting pre-defined criteria.
(9) the method further comprising: providing at least one insight based on the identified at least one pattern.
(10) the method further comprising: for each identified at least one pattern: calculating a risk rate; and in response to the calculated risk rate meeting pre-defined criteria, performing an action.

According to another aspect of the presently disclosed subject matter there is provided a computerized system for providing medical data, the system comprising a processing and memory circuitry (PMC) configured to:
receive data indicative of a first medical data pertaining to a first patient, wherein the first medical data includes at least two different medical data types;
generate a unified representation of the received first medical data;
based on the generated unified representation, conduct a search in a database for identifying stored unified representations that are similar to the generated unified representation, according to a similarity criterion, wherein at least one unified representation of the stored unified representations is associated with a second patient, and is generated based on second medical data of the second patient, wherein the second medical data include at least two different medical data types;
identifying at least one similar unified representation;
obtain the medical data associated with the least one similar unified representation; and
provide the obtained medical data.

According to another aspect of the presently disclosed subject matter there is provided a non-transitory computer readable storage medium tangibly embodying a program of instructions that, when executed by a computer, cause the computer to perform a method for generating a data storage including medical data, the method comprising, by a processor and a memory circuitry:
receiving data indicative of a first medical data pertaining to a first patient, wherein the first medical data includes at least two different medical data types;
generating a unified representation of the received first medical data;
based on the generated unified representation, conducting a search in a database for identifying stored unified representations that are similar to the generated unified representation, according to a similarity criterion, wherein at least one unified representation of the stored unified representations is associated with a second patient, and is generated based on second medical data of the second patient, wherein the second medical data include at least two different medical data types;
identifying at least one similar unified representation;
obtaining the medical data associated with the least one similar unified representation; and
providing the obtained medical data.

The system and the non-transitory computer readable storage medium disclosed in accordance with the aspects of the presently disclosed subject matter detailed above can optionally comprise one or more of features (1) to (10) listed above with respect to the method, mutatis mutandis, in any technically possible combination or permutation.

### BRIEF DESCRIPTION OF THE DRAWINGS

In order to understand the invention and to see how it can be carried out in practice, embodiments will be described, by way of non-limiting examples, with reference to the accompanying drawings, in which:

FIG. 1 illustrates one example of a method for generating a database including medical data, in accordance with certain embodiments of the presently disclosed subject matter.

FIG. 2 illustrates a functional diagram of medical data storage and retrieval system 200, in accordance with certain embodiments of the presently disclosed subject matter;

FIG. 3 illustrates an example of storage 220 included in system 200, in accordance with certain embodiments of the presently disclosed subject matter;

FIG. 4 illustrates a generalized flow chart of a computerized method for generating a data storage including medical data, in accordance with certain embodiments of the presently disclosed subject matter;

FIG. 5 illustrates a generalized flow chart of additional operations performed in generating the data storage, in accordance with certain embodiments of the presently disclosed subject matter; and

FIG. 6 illustrates a generalized flow chart of a computerized method for providing medical data, in accordance with certain embodiments of the presently disclosed subject matter.

### DETAILED DESCRIPTION

In the following detailed description, numerous specific details are set forth in order to provide a thorough understanding of the invention. However, it will be understood by those skilled in the art that the presently disclosed subject matter may be practiced without these specific details. In other instances, well-known methods, procedures, components and circuits have not been described in detail so as not to obscure the presently disclosed subject matter.

Unless specifically stated otherwise, as apparent from the following discussions, it is appreciated that throughout the specification discussions utilizing terms such as “configuring”, “receiving”, “generating”, “storing”, “retrieving”, “determining”, “selecting”, “applying”, “fusing”, “processing”, “associating”, “indexing”, “obtaining”, “conducting”, “searching”, “identifying”, “retrieving”, “providing”, “calculating”, “filtering out”, “applying”, “performing”, or the like, refer to the action(s) and/or process(es) of a computer that manipulate and/or transform data into other data, said data represented as physical, such as electronic, quantities and/or said data representing the physical objects. The term “computer” should be expansively construed to cover any kind of hardware-based electronic device with data processing capabilities including a personal computer, a server, a computing system, a communication device, a processor or processing unit (e.g. digital signal processor (DSP), a microcontroller, a microprocessor, a field programmable gate array (FPGA), an application specific integrated circuit (ASIC), etc.), and any other electronic computing device, including, by way of non-limiting example, computerized systems or devices such as medical system 200 and user workstation 240 disclosed in the present application.

The terms “non-transitory memory” and “non-transitory storage medium” used herein should be expansively construed to cover any volatile or non-volatile computer memory suitable to the presently disclosed subject matter.

Usage of conditional language, such as “may”, “might”, or variants thereof, should be construed as conveying that one or more examples of the subject matter may include, while one or more other examples of the subject matter may not necessarily include, certain methods, procedures, components and features. Thus such conditional language is not generally intended to imply that a particular described method, procedure, component or circuit is necessarily included in all examples of the subject matter. Moreover, the usage of non-conditional language does not necessarily imply that a particular described method, procedure, component, or circuit is necessarily included in all examples of the subject matter. Also, reference in the specification to “one case”, “some cases”, “other cases”, or variants thereof, means that a particular feature, structure or characteristic described in connection with the embodiment(s) is included in at least one embodiment of the presently disclosed subject matter. Thus the appearance of the phrase “one case”, “some cases”, “other cases” or variants thereof does not necessarily refer to the same embodiment(s).

The operations in accordance with the teachings herein may be performed by a computer specially constructed for the desired purposes, or by a general-purpose computer specially configured for the desired purpose by a computer program stored in a non-transitory computer-readable storage medium.

Embodiments of the presently disclosed subject matter are not described with reference to any particular programming language. It will be appreciated that a variety of programming languages may be used to implement the teachings of the presently disclosed subject matter as described herein.

It is appreciated that certain features of the presently disclosed subject matter, which are, for clarity, described in the context of separate embodiments, may also be provided in combination in a single embodiment. Conversely, various features of the presently disclosed subject matter, which are, for brevity, described in the context of a single embodiment, may also be provided separately, or in any suitable sub-combination.

As technology and medical imaging devices evolve, the sources for obtaining medical data, in addition to medical records of patients, are increasing, and include ever-growing medical data available for professionals. However, accessing the tremendous amount of medical data that is now available, in a useful manner, encounters many problems. The sources of medical data include various types, such as the medical records of a patient, including patient parameters including age, sex and history of diseases, history of free text summaries of doctors visits, medical tests such as blood tests or other lab results, and imaging data in various modalities such as MRI, CT, X-Ray. Known methods of searching the data do not support the multiple types of sources of medical data. More specifically, even if the patient data of the various types is gathered, searching the data remains separate for each type of data. Accordingly, existing search tools that are keyword-based, leave the visual/imaging sources of medical data unsearched. Separately operating systems that do searches and retrieve imaging data, provide results with respect to the imaging data with no relation to other medical data of a patient which originated from other non-imaging sources. Also, these systems do not even enable searching the images efficiently, as a search is not performed by searching for a specific pathology, but in a more general visual way, such as tracking the shape of the lungs for example, and not the presence of cancer in the lungs. Such a visual search provides results which are less accurate at best, and sometimes are not relevant at all to the pathology that is being searched.

When professionals are faced with a clinical case, they wish to rely on as much data as possible, as long as it is relevant to the current pathology that has to be diagnosed. Even if professionals wish to rely upon external medical sources, the separate existence of medical sources according to their different types, means that professionals have to do manual data searches, by searching for each type of data separately.

With the growing amount of medical data for each patient, and for all patients as a medical data source, this process becomes non-feasible, resulting in medical data remaining inaccessible. This results in lack of reliance on available medical data, and overlooking potential diagnoses.

Alongside the above medical data based on patients' records, another source of medical data includes medical literature, such as journals and books, which gather data pertaining to unspecified patients, and can include several types of data, in a similar manner to that of a patient record, including imaging data and text, or other numeric data descriptive of the patient's medical condition and identified pathologies. However, professionals also lack the ability to search the medical literature in an efficient manner, for similar reasons as above, i.e., search engines consider only one type of medical data, a keyword-based search, or a visual shape search, and do not gather all types of medical data in a manner which can be searched. Accordingly, any results on searches are inaccurate when searching for pathologies, and results reflect taking into consideration only one type of medical data of the patient, at best.

For illustration only, assume a patient with Appendicitis. His medical records include basic parameters of the patient such as age, gender, and BMI. The medical record further includes several blood test lab results indicating a high degree of white cells count, a CT (or other imaging data) and numerous summaries of doctor visits.

Searching the medical records of this patient, in known systems, enable at most to search the parameters separately, resulting in the patient being also suitable for professionals that search pathology of a different type, such as Inflammatory bowel disease (IBD), which results in a high degree of white cells count as well. Or an Appendicitis with size at the normal size limit, indicative of appendicitis only if accompanied by a high white cell count and appropriate symptoms. However, any search which is based on both types of medical data, which aims to retrieve the above patient only for professionals that seek to search Appendicitis the pathology, or that seek for information on patients which have similar patient parameters, lab results, and imaging data to that stored above for the stored patient, cannot be applied.

It is therefore required to enable to aggregate the different types of the medical data in a single, unified representation, in a manner that enables to search the data such that all patient medical data is considered, irrespective of its data types and the fact that various data types exist. Also, it is required to enable to receive new medical data of a patient, and to be able to aggregate the patient medical data, such that it is possible to compare the new medical data to stored medical data and find similarities, i.e., similar medical data which can assist in providing additional data, insights, and recommendations on treatments to the professional that searches the data.

As explained above, currently, to some extent, AI based systems are being used for diagnosis purposes. However, the black box algorithm-based nature of AI based systems are unexplainable and cannot provide further data to support the diagnosis. It is therefore required to provide a solution that is more explainable, such that if a diagnosis is provided, for example, for a specific cancer, then the professional can go back to the cases, based on which the diagnosis was made, and review the medical records of these patients.

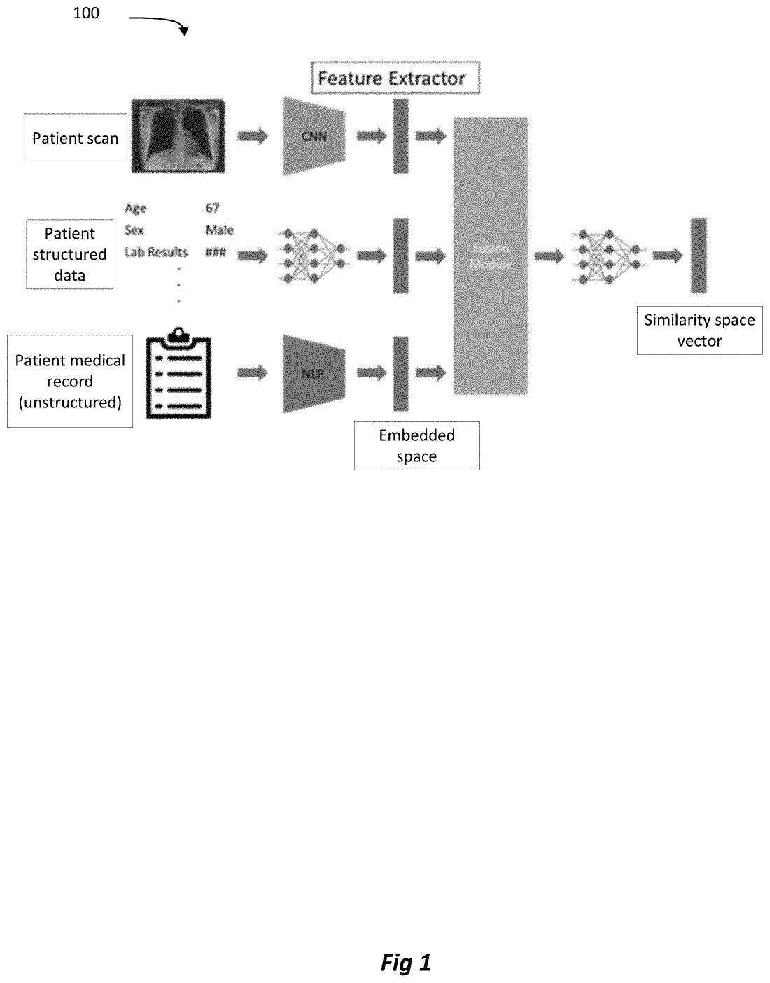

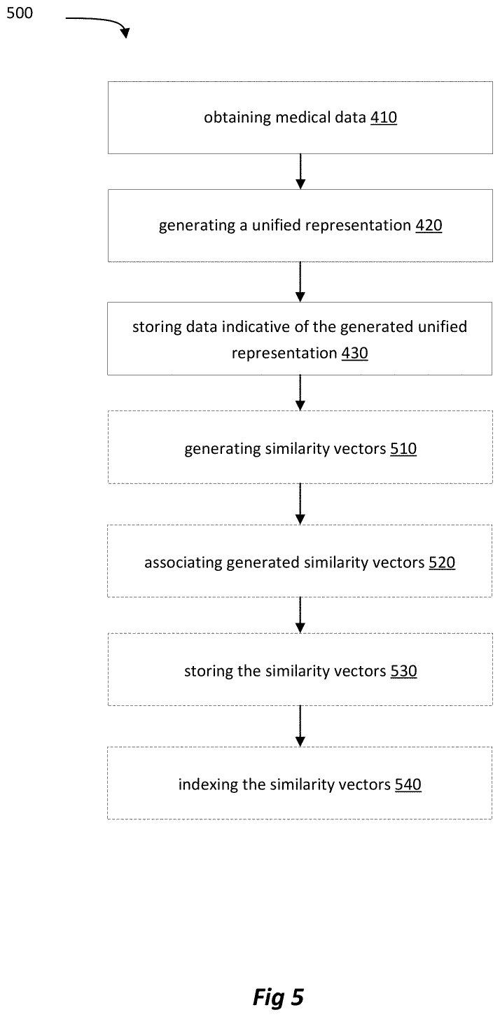

Bearing this in mind, attention is drawn to FIG. 1 illustrating one example of a method for generating one record in a database including medical data, in accordance with certain embodiments of the presently disclosed subject matter. FIG. 1 illustrates an example in which data items pertaining to a patient includes a plurality of different data types. The medical data includes at least two different medical data types. In this example, the medical data comprises an imaging type data including the patient X-ray scan, a second type of medical data comprising patient structured data, such as patient parameters of age, sex, lab results, and a third type of medical data comprising patient medical records, including unstructured data such as summaries of doctor's visits. Each type of medical data is processed, to generate a unified representation of the patient medical data, which is then stored in the medical data. As illustrated in this example, each type of medical data is processed using an AI model, to extract features of the data, to generate a feature vector, uniform to all medical data. The imaging data is processed using a convolutional neural network (CNN) AI model to generate an imaging feature vector, the structured data is processed using another neural network (NN) tool to generate a structured feature vector, and the unstructured data is processed using NLP to generate an unstructured feature vector. The features vectors are then fused using a fusion module to generate the unified representation, e.g., a high dimensional vector which is then stored in the database. The generation of feature vectors from the medical data, having different medical data types, is advantageous, as it enables to generate a single unified representation vector from the medical data of the different types. The single unified representation vector can be generated due to processing each of the different types of data into a uniform format of data, e.g., the feature vectors. FIG. 1 further illustrates that the unified representation is then further processed, e.g., using NN model to generate a similarity vector. The similarity vector is indicative of key features of the patient medical data and can ease the search and retrieval of the data from the database. Further details of the similarity vector appear in FIG. 5.

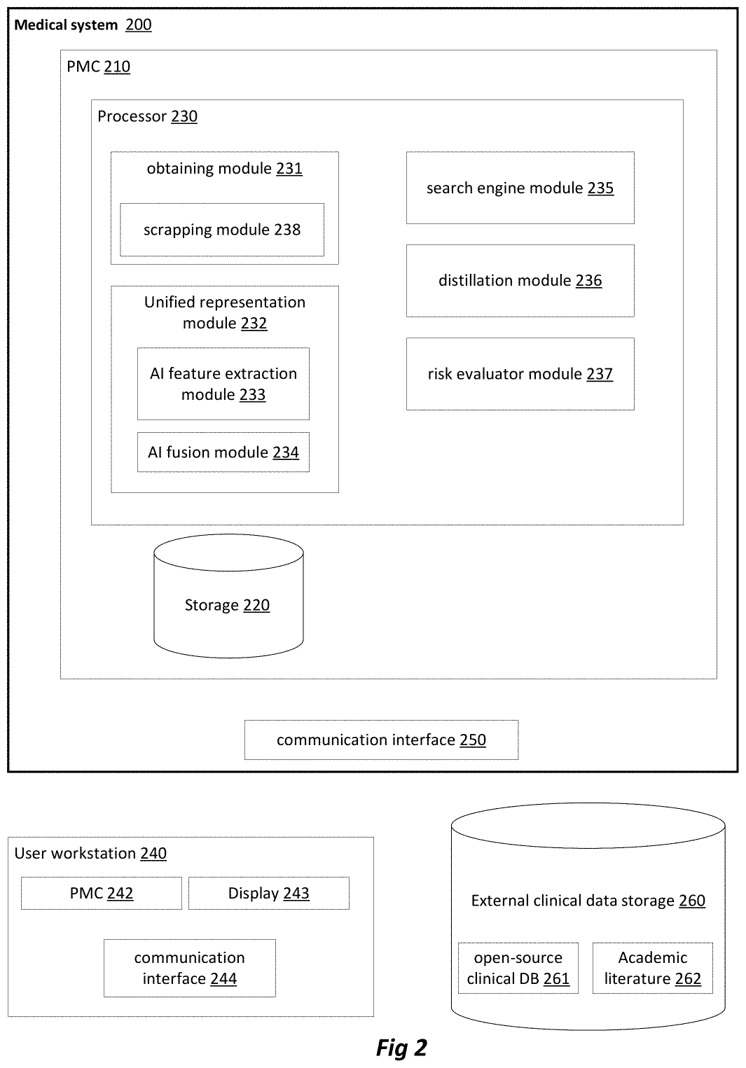

Reference is now made to FIG. 2 illustrating a functional diagram of medical data storage and retrieval system 200, in accordance with certain embodiments of the presently disclosed subject matter. The illustrated medical system 200 comprises several components which operatively communicate with each other, and is configured to store medical data and to enable to a user to retrieve the stored medical data. The medical system 200 comprises a processor and memory circuitry (PMC) 210 comprising a processor 230 and a memory 220 (illustrated as storage 220). Medical system 200 further comprises a communication interface 250 enabling the medical system 200 to operatively communicate with external devices and storages, such as user workstation 240 and external clinical data storage 260. The processor 230 is configured to execute several functional modules in accordance with computer-readable instructions implemented on a non-transitory computer-readable storage medium. Such functional modules are referred to hereinafter as comprised in the processor 230. The processor 230 can comprise an obtaining module 231, a unified representation module 232 that may comprise an AI feature extraction module 233, and an AI fusion module 234. Unified representation module 232 is configured to transform the medical data of the different types, and to generate a high-dimensional numerical representation, as explained below, e.g. using AI modules. The generated high-dimensional numerical representation encodes a large series of numbers that encapsulate information on the medical condition of the patient.

Obtaining module 231 is configured to obtain medical data, for example, from patient medical records such as local clinical data storages, or to scan open databases, such as the Internet and obtain medical literature. Obtaining module 231 can comprise scrapping module 238, further described below.

The medical data obtained by obtaining module 231 can be used by unified representation module 232 to generate unified representation vectors of the medical data. Unified representation vectors can be generated by the feature extraction module 233 including a plurality of AI models configured for processing the medical data into feature vectors, and by AI fusion module 234 configured to fuse the feature vectors into a unified representation vector. The generated unified representation vector can be stored in storage 220.

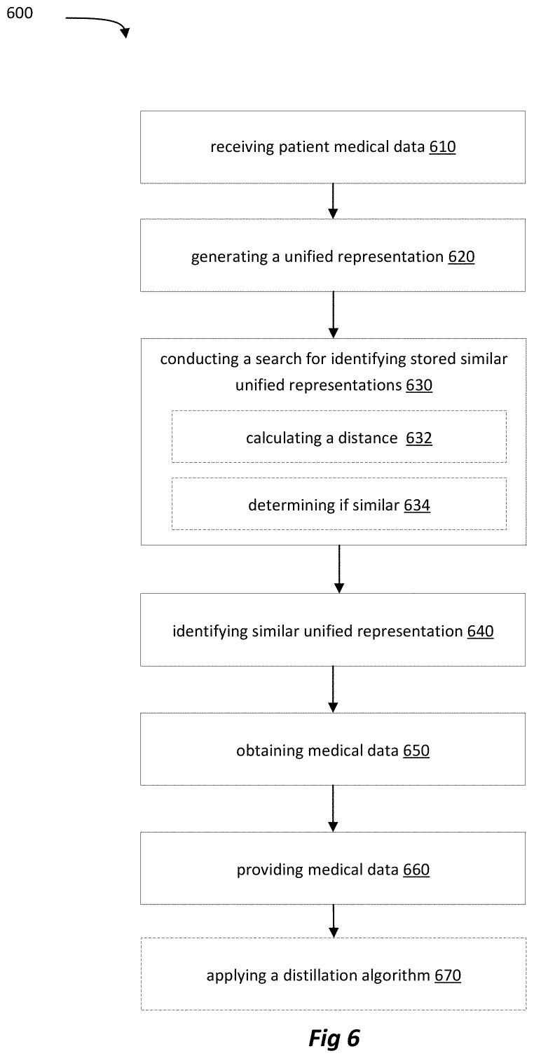

The processor 230 can further comprise a search engine module 235, a distillation module 236, and a risk evaluator module 237. The search engine module 235 is configured to receive new patient medical data, to use unified representation module 232 to generate a unified representation vector of the new data, and to search storage 220 for providing similar medical data. Searching the storage 220 for identifying similar medical data is further described below with respect to FIG. 6. Once similar medical data is identified, distillation module 236 and risk evaluator module 237 are configured to provide further input and processing of the identified data. Further details of the operation of these modules appear with respect to FIG. 6.

FIG. 1 further illustrates user workstation 240 which can be operated by a user (not illustrated), such as a professional. The user can communicate with medical system 200 using the user workstation 240. The user workstation 240 can comprise several components which operatively communicate with each other such as a processor and memory circuitry (PMC) 242 comprising a processor and a memory (not illustrated), a display 243, and a communication interface 244. The user can operate user workstation 240 to communicate, using communication interface 244, new medical data pertaining to the patient, to medical system 200 for processing the new medical data and retrieval of similar medical data stored in storage 220. The results of the similar medical data can be communicated back to the user at user workstation 240 and displayed on display 243.

FIG. 1 further illustrates external clinical data storage 260, comprising clinical database 261, including medical data available to the public, (open source clinical database or open-source clinical database) and academic literature 262 storing medical articles and books including information on certain pathologies. Obtaining module 231 in PMC 210 is configured to obtain data from both of open-source clinical database 261 and the academic literature 262. Medical data included in the open-source clinical database 261 can be obtained using scrapping module 238 included in obtaining module 231. Scrapping module 238 is configured to scrap and identify relevant medical papers and reviews in the academic literature, and to enrich the local data with open-source images, as well as clinical textual and numerical information. The scrapping module 238 may be activated periodically and may look for recently published papers in order to enrich the existing data that has already been scrapped. This process may be conducted periodically by scrapping large academic data sources such as Pubmed.org, Google Scholar, etc. The medical data in the storage 260 can comprise medical data of at least two different medical data types. For example, an article can comprise images of pathologies of a patient along with other data pertaining to the patient, such as patient parameters or patient lab tests. While the article includes patient data, the identity of the patient is unknown and is referred to herein as unspecified patient data. The medical data included in the article can be processed, in a similar manner to processing medical data of a patient, using unified representation module 232, to generate unified representations from the data, and to store the generated unified representations in storage 220.

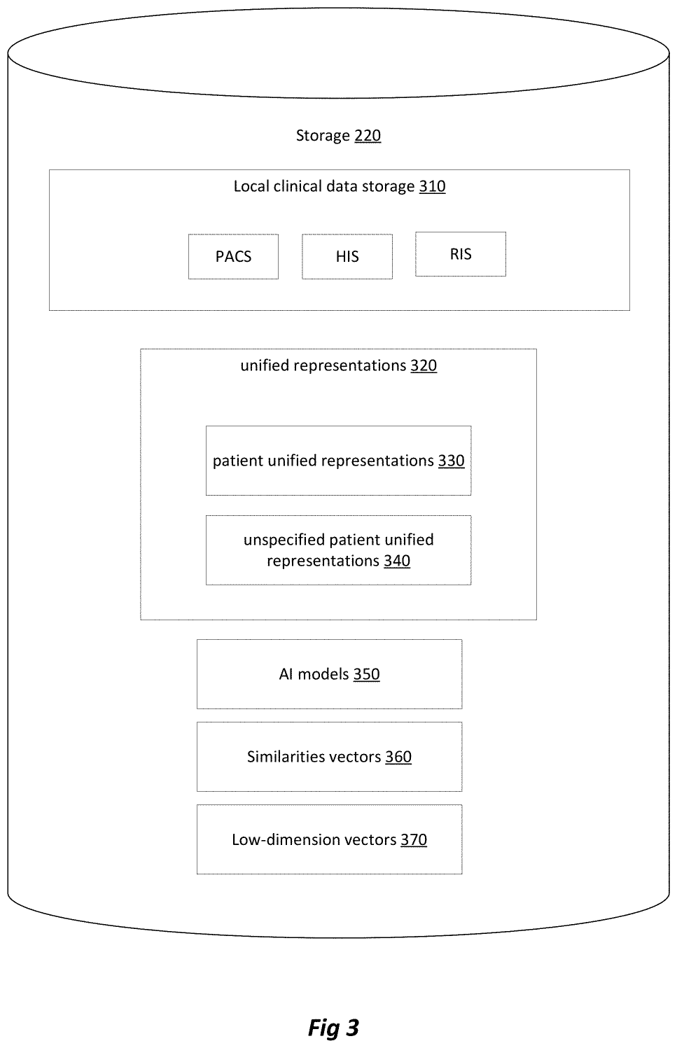

Reference is now made to FIG. 3 illustrating an example of storage 220 included in system 200, in accordance with certain embodiments of the presently disclosed subject matter. System 200 comprises processor 230 for configuring storage 220 to store medical data. Storage 220 may store local clinical data storage 310 comprising medical databases such as Laboratory Information System (LIS), Radiology Information System (RIS), Enterprise Content Management Systems (ECM), Electronic Medical Record (EMR), Hospital Information System (HIS), Picture Archiving and Communication System (PACS), Vendor Neutral Archive (VNA), EMR data, Health Information Exchange (HIE) servers, and others. Illustrated in FIG. 3 are exemplary PACS (picture archive computational systems), HIS (health information system), and RIS (radiologic information system). The first may contain the visual data of all patients registered in the particular medical institute, the second may consist of the radiologic corresponding data of the same registered patients, and the third may contain medical history in the form of textual information and numerical data of lab results of the registered patients. A person having ordinary skills in the art would realize that the above examples are non-limiting, and other information sources may be applicable to the presently disclosed subject matter.

Unified representation module 232 is configured to process data obtained from the local clinical data storage 310 to generate unified representations. Generating unified representations based on local storage may be advantageous, as the local storage may include medical data of a particular type, e.g., of patients who are treated with certain medical equipment which exists only in a specific hospital. In such a manner, system 200 stores medical data relevant to the equipment that is available at that hospital. The generated unified representations based on the local clinical data storage 310 can be stored in unified representations 320, e.g., in patient unified representations 330. Unified representations generated based on open-source clinical DB 261 can also be stored in patient unified representations 330, in a similar manner to unified representations generated based on local clinical data storage.

Unified representations generated based on academic literature 262 can be stored in the unified representations 320, e.g., in unspecified patient unified representations 340. Each unified representation in unified representations 320 is associated with medical data pertaining to a patient, constituting patient medical data, either the medical data of the patients if the unified representation 320 was generated based on patient records, or the unspecified medical data appearing in the academic literature 262 if the unified representation 320 was generated based on academic literature 262.

Storage 220 can further comprise AI models 350 configured to store a plurality of AI models to be used in generating unified representations. Similarities vectors generated based on unified representations can also be stored in storage 220, in similarities vectors 360, wherein each similarity vector is associated with the respective unified representation based on which it was generated, and can further be associated with the respective medical data of the patient associated with the respective unified representation.

In some examples, lower-dimension searchable vectors are generated based on the similarity vectors. Each lower-dimension searchable vector can be associated with the respective similarity vector, and can be stored in low-dimension vectors 370.

It is noted that the teachings of the presently disclosed subject matter are not bound by the medical system 200 described with reference to FIGS. 1-3. Equivalent and/or modified functionality can be consolidated or divided in another manner, and can be implemented in any appropriate combination of software with firmware and/or hardware and executed on a suitable device. The medical system 200 can be a standalone network entity, or integrated, fully or partly, with other network entities. In certain embodiments, one or more components of the medical system 200 can be physically separate from one or more other components and communicate over a data network, and can reside in the cloud on a computer operated by a third party vendor or service provider. Those skilled in the art will also readily appreciate that the data repositories such as databases such as storage 220 and external clinical data storage 260 can be consolidated or divided in other manner, can be shared with other systems, or be provided by other systems, including remote third-party equipment.

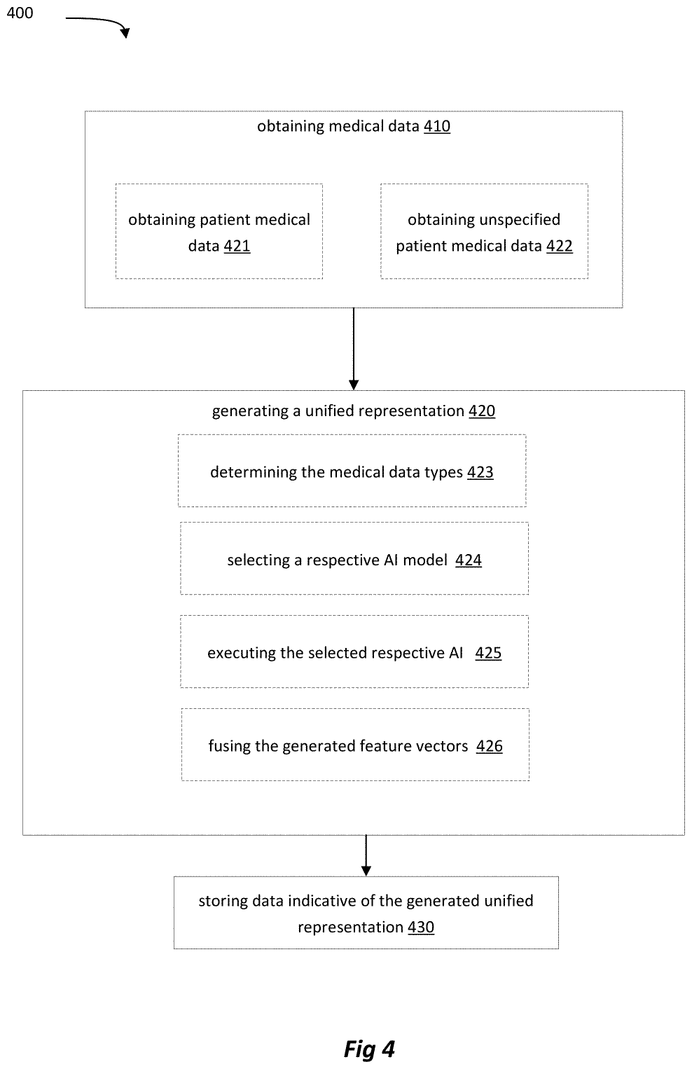

Referring to FIG. 4, there is illustrated a generalized flow chart of a computerized method for generating a data storage including medical data, in accordance with certain embodiments of the presently disclosed subject matter. The following flowchart operations are described with reference to elements of medical system 200, including the PMC 210. However, this is by no means binding, and the operations can be performed by elements other than those described herein.

In some cases, obtaining module 231 can obtain a plurality of data items (block 410). The data items comprise medical data pertaining to a patient, referred to herein as patient medical data. The patient medical data can include at least two different medical data types. For example, a first type of medical data can include different modalities of imaging data including 2D or 3D medical imaging data such as X-Ray, MRI, CT, PET-CT, US, Mammography, Sonography and others. A second type of medical data can include structured data including both text data as well as numeric data, such as patient parameters of age, sex, MRI, lab results, medical tests, patient history, and others. A third type of medical data can include unstructured data such as a doctor's patient summary, patient's clinics summary, patient symptoms and others. The medical data can include a combination of the different types.

In some cases, obtaining module 231 can obtain the medical data from the local clinical data storage 310 in storage 220, e.g. from the local database of a hospital, or from the external clinical data storage 260 or can receive it from user workstation 240, such as if a user communicated medical records into medical system 200. The medical data can include patient medical data such as medical records of specified patients (block 421). Alternatively or additionally, obtaining module 231 can obtain, using scrapping module 238, additional medical information not pertaining to a specified patient, e.g. medical data from academic literature 262 (block 422). Both in the patient medical data, as well as in the academic literature including the unspecified patient medical data, the medical data can include at least two types of the above medical data. In some examples, the medical data includes at least imaging data. Obtaining data from external sources and from the literature, and generating a unified representation which can then be used to identify similarity between vectors, is advantageous, as it enables professionals to ease retrieval of medical data in the same query as searching patient data, where all data considers the different types of medical data, and does not focus on one type only. In many cases, the medical literature is written in a manner which includes imaging data alongside description of other medical data of patients, such that extracting data of different types such as in medical records, based on which the unified representation is generated, is feasible. In some examples, the obtained data can be filtered, for example by removing cases without any imaging data.

Unified representation module 232 using AI feature extraction module 233 can receive the medical data and can process it to generate a unified representation of the obtained patient medical data (block 420). In some examples, AI feature extraction module 233 can determine, for each data, the medical data type (block 423), e.g. whether the data is of imaging type, unstructured data type, or structure data type. In addition, for different modalities of the imaging data, AI feature extraction module 233 can determine whether the imaging data is of CT type or of X-ray type.

Based on the determined medical data type, and optionally, the different modality, AI feature extraction module 233 can select one or more AI models to execute (block 424). The AI models can be selected e.g., from AI models 350 stored in storage 220. For example, the AI models can be selected from a group comprising at least: Convolutional Neural Network (CNN) backbone (ResNet, Nasnet, Inception), Natural Language Processing (NLP), and backbone (RNN, LSTM, Transformers). A person having ordinary skills in the art would realize that other or additional AI models are applicable and can be selected and executed for a data type. In some examples, for each determined type of data, a different respective AI model is selected.

The selected AI model can then be executed for each type of data to generate a feature vector (block 425). The result of executing AI models on a plurality of data items is a plurality of generated feature vectors. For illustration only, referring back to the example of FIG. 1, the imaging data is processed using a CNN AI model to generate an imaging feature vector, the structured data is processed using another neural network (NN) tool to generate a structured feature vector, and the unstructured data is processed using NLP to generate an unstructured feature vector. In some examples, each medical data item having a different medical data type is converted into a uniform representation of a vector type.

AI fusion module 234 can then fuse the resulting feature vectors, to generate a unified representation of the patient medical data (block 426). In some examples, AI fusion module 234 can fuse the feature vectors using an AI fusion model using fusion models known in the art, such as concatenation and attention.

Data indicative of the generated unified representation can be stored in the database (block 430), e.g., in unified representations 320. In some examples, the unified representations are stored. Yet, in some examples, alternatively or additionally, only a subset of the generated unified representations, or a derivative thereof, such as the similarity vectors explained further below, are stored.

Fusing the generated feature vectors into a single unified representation is advantageous, as it enables to aggregate the different types of medical data types into a single, unified representation, which can then be used to compare to other medical data of the same unified representation to identify similarity between unified representations. Comparison between unified representations considers different types of medical data pertaining to a patient, and is advantageous over known systems, where only one type of data, such as textual data type, is compared for identifying similarity. Moreover, if similarity is identified, it is indicative that parameters associated between patients are pathologically similar, where the various types of data are considered.

Referring to FIG. 5, there is illustrated a generalized flow chart of additional operations performed in generating the data storage, in accordance with certain embodiments of the presently disclosed subject matter.

Blocks 410-430 appearing in FIG. 5 correspond to blocks 410-430 appearing in FIG. 4. Once the unified representations are generated and data indicative thereof is stored in storage 220, in some examples some additional operations can be performed, e.g., to facilitate or accelerate future retrieval of data from storage 220. One or more of the unified representations can be processed, e.g., using an AI model, to generate one or more similarity vectors, e.g. using known methods. Each similarity vector may be indicative of key features of the patient medical data (block 510). For example, if the patient's records indicate high white blood cell count, then high white blood cell count is reflected by the corresponding similarity vector.

Each generated similarity vector can be associated with the respective unified representation and/or with the respective patient medical data (block 520) and can be stored in storage 220 (block 530). Future search for similarity of medical data to a new patient data can be performed on the similarity vectors, rather than the unified representations. Searching for similarities based on the similarity vectors rather than on the unified representations is advantageous since search can be conducted in a more accurate manner, as the similarities vectors are generated in such a manner that facilitates comparing two vectors to determine similarity between them.

To further facilitate retrieval of data from storage 220, the similarity vectors can be indexed using one or more of the following indexing methods (block 540). For example, indexing can be performed by associating a similarity vector with one or more predefined searchable data fields from the patient medical data. For example, a parameter lookup tree can be determined, including one or more predefined searchable data fields that are identified in the patient medical data. The future search query on the database will include one or more of the same or similar fields. For example, the fields can include imaging modality (Xray, CT, etc.), ROI organ, etc. The query with the predefined fields can be routed through the lookup tree to search within a smaller subset of the database. Each similarity vector can be associated with one or more fields in accordance with the parameter lookup tree.

Another example of indexing the similarity vectors can include generating a lower-dimension searchable vector, e.g., using dimension reduction lookup. The similarity vector, being a high dimensionality vector, is reduced to a much lower dimension, while encapsulating most of the critical data about the similarity vector. The low dimension similarity vectors can be stored in low-dimension vectors 370 in storage 220. The dimension reduction is advantageous as it facilitates in achieving a much faster similarity calculation between a future query and the stored database similarity vectors. Once similar lower-dimension searchable vectors are identified, a fine search is conducted on the few most similar vectors, to calculate and identify the most accurate similar vectors. The generated lower-dimension searchable vector can be associated with the respective similarity vector.

Any identified similar vectors, including unified representations, similarity vectors, and reduced low dimension vectors, can be provided to the user via user workstation 240. The user can then retrieve the medical records associated with the identified similar vectors for further data of the identified results.

Referring to FIG. 6, there is illustrated a generalized flow chart of a computerized method for providing medical data, in accordance with certain embodiments of the presently disclosed subject matter. The following flowchart operations are described with reference to elements of medical system 200, including the PMC 210. However, this is by no means binding, and the operations can be performed by elements other than those described herein.

Assuming a professional that has a new female patient at the age of 80, experiencing some particular symptoms, such as pain in the breast. The patient already performed a breast mammography (CT imaging data), all recorded in her medical records. The professional would like to retrieve medical data on similar cases of patients, e.g. patients having similar medical data to that of the new female patient. For example, females over the age of 45, who also experienced pain in the breast and had a breast mammography. The professional may communicate using user workstation 240 the medical records of the new patient, or a part thereof, to medical system 200 to retrieve similar stored medical records.

In some cases, obtaining module 200 can receive data indicative of the medical data pertaining to the new patient, constituting a first patient (block 610). The first medical data pertaining to the first patient communicated by the professional includes at least two different medical data types. In the above example, the medical data includes three different types of data: the imaging type including the CT, the structured data including the patient parameters such as age 80, and the symptoms of the patient, pain in the breast. In addition, the medical records also include unstructured data including the professional's visit summary describing the status and reason for visit of the new patient.

In some examples, in a similar manner to that described above with respect to block 420 in FIG. 4, with respect to generating a unified representation of obtained medical data, unified representation module 232 can generate a unified representation for the first medical data of the new patient (block 620). For example, the imaging type data of the CT can be determined (in a similar manner to that described above with respect to block 423), and a respective AI model from stored AI models 350, such as CNN, can be selected and executed (in a similar manner to that described above with respect to blocks 424 and 425). In addition, the structured biopsy data can be identified, and an NN can be applied to generate a respective feature vector. Also, the unstructured type of the professional's visit summary can be determined, and an NLP can be selected and executed. Executing the one or more AI models for each type of data on the new patient results in a plurality of feature vectors. The feature vectors can then be fused, e.g., using an AI fusion model, to generate a unified representation (in a similar manner to that described above with respect to block 426). Generating the unified representation is advantageous as it results in a single unified vector, representing all types of medical data of the new patient. Also, the generated unified representation has a uniform format of data to other stored medical data, pertaining to a plurality of patients, which enable the search for similar medical data.

Therefore, based on the generated unified representation, a search in storage 220 can be conducted by search engine module 235 for identifying stored unified representations that are similar to the generated unified representation (block 630). Searching storage 220 can be done in either or both patient unified representations 330 and unspecified patient unified representations 340. Alternatively, searching can be done in similarities vectors 360 including the respective similarity vectors to the unified representations.

In some examples, identifying whether stored unified representations are similar to the new generated unified representation can be determined in accordance with a similarity criterion. For example, for each stored unified representation of the plurality of stored unified representations, a distance between the new generated unified representation and the stored unified representation can be calculated (block 623). Calculating the distance between the two vectors can be done e.g., using known methods, such as |1, |2, Mahalanobis distance, or other known methods of measuring a distance. The calculated distance can be compared to a pre-configured threshold. If the calculated distance meets low value of a distance metric, such that it does not exceed the pre-configured threshold, it can be determined that the stored unified representation meets the similarity criterion (block 634).

In some examples, a distance can be calculated between the new generated unified representation and each stored unified representation. Each calculated distance can be compared to the pre-configured threshold. If the distance does not exceed the pre-configured threshold, the stored unified representation is determined to be similar to the new unified representation.

The stored unified representation reflects medical data of patients, of different types, where, in some examples, at least one unified representation of the stored unified representations is associated with a second patient, and the unified representation was generated based on second medical data of the second patient. The second medical data includes at least two different medical data types. The second medical data types pertaining to the second patient may be different or partially different to the medical data received for the new patient. For example, the stored unified representation can be based on medical data comprising imaging data type and unstructured data type, whereas the new unified representation can be based on medical data comprising imaging data type and structured data type.

Since the unified representations represent stored medical data of patients, in some examples, those unified representations that are determined to be similar to the new unified representation can be indicative that at least one parameter of the stored medical data has pathology similar to at least one parameter of the new medical data. As described above, the stored unified representations can include similarity vectors generated based on unified representations. As explained above, calculating the distance and determining if the unified representations are similar to the new unified representation can be done based on the similarity vectors stored in similarity vectors 360.

As a result of the search, at least one unified representation can be identified as similar to the new generated unified representation (block 640). Each stored unified representation or stored similarity vector can be associated with medical data based on which they were generated. Once similar vectors are identified, the associated medical data can be obtained, e.g. by retrieving the stored medical data from local clinical data storage 310 (block 650). The medical data can be provided back to the user workstation 240 and can be displayed on display 243 to the professional review (block 660).

The association between the generated unified representations, the similarity vectors, and the original medical records is advantageous, as it enables, once similar vectors are identified, to go back to the original medical records to receive additional information on the similar medical data. Moreover, in some examples, prior to providing the data for professional review, one or more actions can be executed on the identified similar vectors, to improve the results that are provided to the user. For example, a similarity degree can be calculated based on the calculated distance, where the similarity degree is indicative of a degree that the stored and the new vectors are similar. The medical data that is retrieved can be provided along with the similarity degree, providing an indication to the user of the similarity to the stored data. Alternatively, the identified similar unified representation can be prioritized based on the similarity degree. In some examples, identified similar unified representations having a higher priority will be provided first to the user, resulting in a more efficient manner of retrieval of the medical data from the storage 220.

Yet, in some examples, prior to obtaining the stored medical data, at least one identified similar unified representation can be filtered out, based on medical heuristics. For example, medical heuristics can include that any identified similarity of a 70 year old man and a 1-year old baby will be drastically reduced, resulting in filtering out identified vectors pertaining to people who are below a certain age, e.g. based on the difference in years between the new patient and the identified similar vectors of stored patients. The stored medical data of similar unified representation which were not filtered out can then be obtained. Additional examples include that a stored breast cancer similarity vector will not be compared with a query for a male patient. Also, a disease that medically has to present a high white blood cell count will not be compared with a low white blood cell count query for a patient. A person versed in the art would realize that other heuristics may be applied in accordance with the presently disclosed subject matter.

In some examples, the professional providing the medical data of the new patient can further provide a region of interest (ROI) to search. In such cases, receiving the patient medical data as described above in block 610 further comprises receiving a user input with respect to an ROI. For example, the ROI can be the professional marking on the imaging medical data, indicating a specific region to be searched. The user input can be used by obtaining module 231 to define the medical data inputted to the unified representation module 232. In the example of the marking of the professional on the imaging data, the marked portion of the imaging data can be used to generate the unified representation, as opposed to the entire image.

Receiving additional input from the user, such as in the form of ROI, is advantageous, since it facilitates focusing the search and providing additional information to system 200. The additional information is reflected by more focused imaging data provided to system 200, such that the unified representation later generated, based on the focused imaging data, is then searched. The stored unified representations which are then identified as similar to new unified representation are more likely to include medical data that is clinically similar to the focused area marked by the professional, thereby resulting in a more efficient retrieval of similar medical data from system 200.

In some examples, once a plurality of similar unified representations are identified, a distillation algorithm can be executed, e.g. by distillation module 236, to facilitate the efficiency of the medical data retrieved from system 220 (block 670). The distillation algorithm can provide additional data based on the identified similar vectors and the medical records associated with the identified similar vectors such as patterns. As explained further below, patterns can include diagnosis, possible consequences and suggested treatment.

In some examples, a pattern based on the associated similar medical data can be determined. A pattern can be for example the following derivative data identified in the medical records:
1. Diagnosis such as a certain pathology (e.g. a lung cancer)
2. Consequences: e.g. certain rate of patients that experienced a level of mortality
3. Treatment: certain rate of patients that had a same treatment, e.g. chemo.

As an example, a pattern can be a common treatment for the distilled pathology, for example, a patient with liver cancer would need dissection with chemo (e.g. if all or the majority of the similar patients based, on the similar vectors, received this treatment. Hence, dissection with chemo may be the identified pattern). Pattern can also be a consequence of identifying the common consequences of all of the similar patients. A person versed in the art would realize that other types of patterns are also applicable in the presently disclosed subject matter. In some examples, a pattern can be associated, in advanced, with medical data pertaining to a patient, and, accordingly, associated with the stored unified representations or the stored similarity vectors. However, this is not binding, and a pattern can be identified e.g., in real time, using an NLP algorithm executed on the medical data, once the similarity vectors are identified. In some examples, once at least a first and second similar unified representations (which can be the similarity vectors themselves) are identified as similar to the new unified representation, statistical methods can be applied on the identified first and second respective medical records associated with the identified unified representations to identify at least one pattern in the associated medical data. For each identified pattern, a respective probability can be calculated, e.g. based on the statistical significance of the pattern. To illustrate, in one simplified manner, statistical significance can include the number of patients with the identified pattern among the overall number of patients. In more exemplified advanced calculations, other factors may be taken into consideration. For example, the similarity degree can be taken into consideration, e.g., the most similar to the retrieved patient—the most significant the patient is to the calculation (for example, multiply the contribution to the identified pattern with a factor decreasing from 1 to 0, depending on how similar the patient is (the distance between the vectors). Patient data trust level can also be taken into consideration—each retrieved patient contribution will be multiplied by the trust level of the extracted data of this patient (high trust level—factor of 1—remain with high contribution to the identified pattern, low trust level—factor of near 0—contribution to the pattern is drastically decreased). Other examples are also applicable to the presently disclosed subject matter. The highest identified pattern, along with its probability, can be provided to the user. Additionally, for each calculated probability, it can be determined whether it meets pre-defined criteria, and if so, the pattern along with its probability can be provided to the user. For example, pre-defined criteria can include percentage of the highest probabilities, probabilities that exceeds a pre-defined threshold, etc.

In some examples, based on the pattern, at least one insight can further be provided to the user, along with the identified pattern. For example, the following corresponding insights can be provided to the user based on the above identified patterns:
1. Diagnosis: 70% of the results patients (i.e. the patients associated with the identified similar unified representations) had a lung cancer (e.g., their medical data indicates a pattern of diagnosis—70% lung cancer)
2. Consequences: 60% of the above 70% patients having lung cancer died within 3 years (another pattern of consequences—60% mortality rate in 3 years)
3. Treatment: 90% of them were treated with chemo (another pattern of treatment—chemo).

Such insights can assist the professional to determine treatment, given insights on similar patients.

Identifying a pattern and providing an insight to the user based on the medical data associated with the identified similar vectors is advantageous as the identified pattern is not limited to a pre-defined closed list of pathologies or diagnoses and is relies upon common data found in all identified vectors, once identified as similar to the new patient data.

In some examples, based on an identified pattern, a risk rate can be calculated, by risk evaluator module 237. A diagnosis pattern can be associated with a severity level (e.g., 1-5). For example, breast cancer can be associated with level 5. In a simplified operation of the algorithm, the risk rate can be calculated by the diagnosis pattern probability multiplied by its severity. If the results are above a threshold, the calculated risk rate will be higher. It will be appreciated that other calculations of the risk rate can be determined, based on the pattern and the severity.

In response to the risk rate meeting a pre-defined criterion, an action can be taken. For example, if the risk rate is high, also due to the severity of the pattern, then a suitable alert is given to the professional who inserted the new medical data to system 200. In addition, the new patient for which data was searched, may be prioritized e.g., in a hospital queue at the hospital triage, or suitable alerts are communicated to the relevant professionals at the hospital who can treat the identified patient. Alternatively, the risk rate, in combination with other parameters of the medical records (age, sex) can be combined and be compared to determine whether they meet a threshold. For example, a certain risk rate would not be considered as having a high priority, however, when combined with an advanced age of a patient, the risk rate increases, such that high priority would be given to an aged patient.

In some examples, the distillation algorithm system and method have one or more intervention modes, e.g., are suitable for the distinct level of expertise: a trainee mode and an expert mode. In some examples, the distillation module 236 may run as a backend service, while processing new patient records inserted into the system. The distillation module 236 may track patient records and the diagnosis inserted by professionals. The distillation module 236 may run constantly while reviewing new medical data inserted into the system 200, or may be triggered once new medical data is inserted.

The distillation module 236 may process the medical data inserted into the system 200 in a similar manner to that described above with respect to steps 610-650, including identifying any patterns which are raised from any similar vectors identified to be similar to the new patient data. The distillation module 236 may provide, to the professional who has inserted the data, other entities relating to the new patient, such as the triage or other data pertaining to the processing of the new medical data. For example, distillation module 236 may display and communicate patterns that were identified, and present a suggested diagnosis and recommended treatment. The provided data can be displayed, e.g., based on priority level, to highlight similar cases that were identified. Distillation module 236 may also prioritize the patient at the triage, e.g., based on the processed data and the identified similar vectors.

In some examples, the distillation module 236 may not provide data in an active ‘push’ manner, but may process the new medical data and intervene, merely to avoid malpractice. In such examples, the distillation module 236 may further process the diagnosis determined by the professional with respect to the new patient e.g., as included in the doctor's summary in the medical records. The diagnosis can be processed in a similar manner to that described above with respect to steps 610-620 using e.g., NLP model, e.g., using unified representation module 232. The professional's diagnosis can then be compared to the identified similar vectors and any patterns that were identified, based on medical data associated with the similar vectors. If the professional's diagnosis is identical or similar to the identified similarity vectors and the patterns that are raised from the similarity vectors (e.g., such that it meets a similarity criterion), then no further action is taken by the distillation module 236. If, on the other hand, the professional's diagnosis and the identified pattern are not similar, then distillation module 236 may intervene and display the identified pattern and/or the medical records associated with the identified pattern, to the professional. In such a manner, possible malpractice may be prevented.

In some examples, a user may manually set the mode at system 200 such that, in a trainee mode, the distillation module 236 operates in an active push manner and provides data to the professional and other entities constantly, while, in an expert mode, the distillation module 236 provides data only in case the processed diagnosis deviates from the processed medical data, patterns, and diagnosis that is identified by system 200.

It should be noted that the term “criterion” or “criteria” as used herein should be expansively construed to include any compound criterion, including, for example, several criteria and/or their logical combinations. Also, the specific examples of criteria should not be considered as limiting, and those skilled in the art will readily appreciate that the teachings of the presently disclosed subject matter are, likewise, applicable to other criteria.

It is noted that the teachings of the presently disclosed subject matter are not bound by the flow chart illustrated in FIGS. 4-6, and that the illustrated operations can occur out of the illustrated order. For example, operations 430, and 510-540, shown in succession, can be executed substantially concurrently, or in the reverse order.

It is to be understood that the invention is not limited in its application to the details set forth in the description contained herein or illustrated in the drawings. The invention is capable of other embodiments and of being practiced and carried out in various ways. Hence, it is to be understood that the phraseology and terminology employed herein are for the purpose of description and should not be regarded as limiting. As such, those skilled in the art will appreciate that the conception upon which this disclosure is based may readily be utilized as a basis for designing other structures, methods, and systems for carrying out the several purposes of the presently disclosed subject matter.

It will also be understood that the system according to the invention may be, at least partly, implemented on a suitably programmed computer. Likewise, the invention contemplates a computer program being readable by a computer for executing the method of the invention. The invention further contemplates a non-transitory computer-readable memory tangibly embodying a program of instructions executable by the computer for executing the method of the invention.

Those skilled in the art will readily appreciate that various modifications and changes can be applied to the embodiments of the invention as hereinbefore described without departing from its scope, defined in and by the appended claims.

1. A computerized method for generating a data storage including medical data, the method comprising by a processor and a memory circuitry:
obtaining a plurality of data items, wherein the data items comprise medical data pertaining to a patient, constituting patient medical data, wherein the patient medical data includes at least two different medical data types;
processing the plurality of obtained medical data to generate a unified representation of the patient medical data; and
storing data indicative of the generated unified representation in the database.

2. The method of claim 1, wherein the processing is done using one or more AI models.

3. The method of claim 1, wherein generating the unified representation further comprises:
determining the medical data types of the patient medical data;
for each determined data type, selecting a respective AI model to execute on the patient medical data;
for each determined data type, executing the selected respective AI model to generate a feature vector, resulting in a plurality of generated feature vectors;
fusing the generated feature vectors to generate a unified representation of the patient medical data.

4. The method of claim 1, further comprising:
processing the generated unified representation, using an AI model, to generate a similarity vector, wherein the similarity vector is indicative of key features of the patient medical data;
associating generated similarity vector with the unified representation; and
storing the generated similarity vector.

5. The method of claim 4, further comprising:
indexing the similarity vector to facilitate retrieval of the medical data from the memory.

6. The method of claim 5, wherein indexing the similarity vector further comprises:
associating the similarity vector with one or more predefined searchable data fields from the patient medical data or based on the similarity vector, generating a lower-dimension searchable vector and associating the generated lower-dimension searchable vector with the similarity vector.

7. The method of claim 1, further comprising:
obtaining additional medical information not pertaining to a specified patient;
generating a unified representation of the additional medical information; and
storing the generated unified representation in the memory.

8. A medical data storage and retrieval system for a computer having a processing and memory circuit (PMC), comprising:
a processor of the PMC for configuring the memory of the PMC to store medical data, wherein the medical data comprises:
a plurality of unified representations,
wherein each unified representation is associated with medical data pertaining to a patient, constituting patient medical data, and was generated by processing a plurality of data items, wherein the data items comprise medical data, wherein the medical data includes at least two different medical data types.

9. The system of claim 8, wherein each unified representation is generated using one or more AI models.

10. The method of claim 1, wherein the patient medical data comprises at least two of: medical records including unstructured or structured data, 2D or 3D medical imaging data, medical tests, patient history, a doctor's patient summary, patient's clinics summary, or a combination thereof.

11. The system of claim 8, wherein each of the unified representations is generated by:
determining the medical data types of the patient medical data;
for each determined data type, selecting a respective AI model to apply on the patient medical data;
for each determined data type, applying the selected respective AI model to generate a feature vector, resulting in a plurality of generated feature vectors;
fusing the generated feature vectors to generate the unified representation of the patient medical data.

12. The method of claim 1, wherein the AI models are selected from a group comprising at least: Convolutional Neural Network (CNN) backbone, Fully Connected Network (FCN), and NLP (Natural Language Processing) backbone.

13. The method of claim 1, wherein fusing the generated feature vectors is performed by an AI fusion model.

14. The system of claim 8, wherein each of the unified representations is associated with a respective similarity vector, wherein each similarity vector is generated from the unified representation, using an AI model, and is indicative of key features of the patient medical data.

15. The system of claim 14, wherein the similarity vectors are indexed to facilitate retrieval of the medical data from the memory.

16. The system of claim 15, wherein each similarity vector is associated with one or more predefined searchable data fields from the patient medical data.

17. The system of claim 15, wherein each similarity vector is associated with a generated lower-dimension searchable vector.

18. The system of claim 8, wherein the medical data further comprises:
a plurality of unspecified patient unified representations;
wherein each unspecified patient unified representation is associated with additional medical information not pertaining to a specified patient, and is generated based on the medical information not pertaining to a specified patient, wherein the additional medical information includes at least two different medical data types.

19. A non-transitory computer readable storage medium tangibly embodying a program of instructions that, when executed by a computer, cause the computer to perform a method for generating a data storage including medical data, the method comprising, by a processor and a memory circuitry:
obtaining a plurality of data items, wherein the data items comprise medical data pertaining to a patient, constituting patient medical data, wherein the patient medical data includes at least two different medical data types;
processing the plurality of obtained medical data to generate a unified representation of the patient medical data; and
storing data indicative of the generated unified representation in the database.
